(19) United States
(12) Patent Application Publication
(10) Pub. No.: US 2025/0318843 A1
(43) Pub. Date: Oct. 16, 2025

(54) SYSTEMS, DEVICES, AND METHODS FOR TREATING VASCULAR OCCLUSIONS

(71) Applicant: Inari Medical, Inc., Irvine, CA (US)

(72) Inventor: James Quang Dinh, Irvine, CA (US)

(21) Appl. No.: 19/066,855

(22) Filed: Feb. 28, 2025

Related U.S. Application Data
(63) Continuation of application No. 18/443,866, filed on Feb. 16, 2024, now Pat. No. 12,274,459.
Application No. 18/443,866 is a continuation of application No. 18/351,326, filed on Jul. 12, 2023, now Pat. No. 11,937,834.
Application No. 18/351,326 is a continuation of application No. 17/072,909, filed on Oct. 16, 2020, now Pat. No. 11,864,779.
(60) Provisional application No. 62/916,044, filed on Oct. 16, 2019.

(51) Int. Cl. A61B 17/22; A61B 17/00

(52) U.S. Cl. CPC A61B 17/22; A61B 2017/00867; A61B 2017/22038; A61B 2017/22079

(57) ABSTRACT

Systems and methods for the intravascular treatment of clot material within a blood vessel of a human patient are disclosed herein. A device in accordance with embodiments of the present technology can include, for example, a plurality of interconnected struts forming a unitary structure having a proximal portion and a distal portion. The struts can form a plurality of first cells in the proximal portion and a plurality of second cells, smaller than the first cells, in the distal portion. The device can be pulled against clot material within a blood vessel to engage, disrupt, and/or capture the clot material.

### CROSS-REFERENCE TO RELATED APPLICATION

This application is a continuation of U.S. patent application Ser. No. 18/443,866, filed Feb. 16, 2024, and titled “SYSTEMS, DEVICES, AND METHODS FOR TREATING VASCULAR OCCLUSIONS,” which is a continuation of U.S. patent application Ser. No. 18/351,326, filed Jul. 12, 2023, and titled “SYSTEMS, DEVICES, AND METHODS FOR TREATING VASCULAR OCCLUSIONS,” issued as U.S. Pat. No. 11,937,834, which is a continuation of U.S. patent application Ser. No. 17/072,909, filed Oct. 16, 2020, and titled “SYSTEMS, DEVICES, AND METHODS FOR TREATING VASCULAR OCCLUSIONS,” issued as U.S. Pat. No. 11,864,779, which claims the benefit of U.S. Provisional Patent Application No. 62/916,044, filed Oct. 16, 2019, and titled “SYSTEMS, DEVICES, AND METHODS FOR TREATING VASCULAR OCCLUSIONS,” each of which is incorporated herein by reference in its entirety.

### TECHNICAL FIELD

The present technology relates generally to systems, devices, and methods for the intravascular treatment of colt material (e.g., emboli and/or thrombi) within a blood vessel of a human patient. In particular, some embodiments of the present technology relate to expandable devices for engaging and removing clot material.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by a clot, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long distance air travel, immobility, etc.) and clotting (e.g., cancer, recent surgery, such as orthopedic surgery, etc.). DVT can obstruct drainage of venous blood from the legs leading to swelling, ulcers, pain and infection. DVT can also create a reservoir in which blood clots can collect and then travel to other parts of the body including the heart, lungs, brain (stroke), abdominal organs, and/or extremities.

In the pulmonary circulation, the undesirable material can cause harm by obstructing pulmonary arteries-a condition known as pulmonary embolism. If the obstruction is upstream, in the main or large branch pulmonary arteries, it can severely compromise total blood flow within the lungs, and therefore the entire body. This can result in low blood pressure and shock. If the obstruction is downstream, in large to medium pulmonary artery branches, it can prevent a significant portion of the lung from participating in the exchange of gases to the blood resulting in low blood oxygen and buildup of blood carbon dioxide.

There are many existing techniques to reestablish blood flow through an occluded vessel. Embolectomies, for example, are a surgical technique involving incising a blood vessel and placing a balloon-tipped device (such as the Fogarty catheter) at the location of the occlusion. The balloon is then inflated at a point beyond the clot and used to withdraw the obstructing material back to the point of incision. The obstructing material is then removed by the surgeon. Although such surgical techniques have been useful, exposing a patient to surgery may be traumatic and best avoided when possible. Additionally, the use of a Fogarty catheter may be problematic due to the possible risk of damaging the interior lining of the vessel as the catheter is being withdrawn.

Percutaneous methods are also utilized for reestablishing blood flow. A common percutaneous technique is referred to as balloon angioplasty where a balloon-tipped catheter is introduced to a blood vessel (e.g., typically through an introducing catheter). The balloon-tipped catheter is then advanced to the point of the occlusion and inflated to dilate the stenosis. Balloon angioplasty is appropriate for treating vessel stenosis, but it is generally not effective for treating acute thromboembolisms as none of the occlusive material is removed and restenosis regularly occurs after dilation. Another percutaneous technique involves placing a catheter near the clot and infusing streptokinase, urokinase, or other thrombolytic agents to dissolve the clot. Unfortunately, thrombolysis typically takes hours to days to be successful. Additionally, thrombolytic agents can cause hemorrhage, and in many patients the thrombolytic agents cannot be used at all.

Various devices exist for performing a thrombectomy or removing other foreign material. However, such devices have been found to have structures which are either highly complex, cause trauma to the treatment vessel, or lack the ability to be appropriately fixed against the vessel. Furthermore, many of the devices have highly complex structures that lead to manufacturing and quality control difficulties as well as delivery issues when passing through tortuous or small diameter catheters. Less complex devices may allow the user to pull through the clot, particularly with inexperienced users, and such devices may not completely capture and/or collect all of the clot material.

Thus, there exists a need for improved systems and methods for embolic extraction.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIGS. 1A and 1B are side views of a clot treatment system in a pre-deployed configuration and a deployed configuration, respectively, configured in accordance with embodiments of the present technology.

FIG. 1C is an enlarged perspective view of a distal portion of the clot treatment system shown in FIG. 1B configured in accordance with an embodiment of the present technology.

FIGS. 2A-2C are a side view, a proximally-facing perspective view, and a distally-facing perspective view, respectively, of a clot treatment device of the clot treatment system of FIGS. 1A-1C configured in accordance with embodiments of the present technology.

FIG. 3 is a flow diagram of a process or method for operating the clot treatment system to remove clot material from within a blood vessel of a human patient in accordance with an embodiment of the present technology.

FIGS. 4A-4F are schematic illustrations of a distal portion of the clot treatment system during a procedure to remove clot material from a blood vessel of a human patient in accordance with embodiments of the present technology.

### DETAILED DESCRIPTION

The present technology is generally directed to systems, devices, and methods for removing clot material from a blood vessel of a human patient. In some embodiments, a clot removal system can include a delivery catheter and a clot treatment device. The clot treatment device can include a plurality of interconnected struts forming a unitary structure that is movable between a compressed configuration and an expanded configuration. In the expanded configuration, the unitary structure can include (i) a proximal connection region, (ii) a proximal conical region extending from the proximal connection region, (iii) a cylindrical region extending from the proximal conical region, (iv) a distal conical region extending from the cylindrical region, and (v) a distal connection region extending from the distal conical region. In some embodiments, a first portion of the struts form first cells in the proximal conical region, and a second portion of the struts form second cells in the distal conical region that are smaller than the first cells.

In some embodiments, the system further includes a handle configured to be gripped by an operator, and a first shaft coupled between the handle and the proximal connection region of the clot treatment device. The clot treatment device can be maintained in the compressed configuration within a lumen of the delivery catheter and near a distal terminus of the delivery catheter. To move the clot treatment device to the expanded configuration, the operator can move the handle to advance the first shaft to thereby advance the clot treatment device past the distal terminus and out of the lumen of the delivery catheter. When the clot treatment device is no longer constrained by the delivery catheter, the clot treatment device can expand (e.g., self-expand) to the expanded configuration. In some embodiments, the system further includes a second shaft extending at least partially through the first shaft and coupled to the distal connection region of the clot treatment device. Relative movement between the first and second shafts can allow the clot treatment device to lengthen/shorten and to correspondingly radially expand/compress.

During a procedure to remove clot material from a blood vessel of a human patient, the clot treatment device can be expanded distal of the clot material within the blood vessel, and then retracted proximally into the clot material to capture/disrupt the clot material. In one aspect of the present technology, the larger first cells of the clot treatment device are configured to receive the clot material therethrough as the clot treatment device is pulled against the clot material, and the smaller second cells of the clot treatment device are configured to retain the clot material within the clot treatment device. In another aspect of the present technology, the clot treatment device has sufficient radial stiffness (e.g., at the cylindrical region) to inhibit the clot treatment device from slipping (e.g., not engaging) the clot material when the clot treatment device is pulled against the clot material. Accordingly, the clot treatment device can be used to capture/disrupt adhered, organized, and/or chronic clots that would otherwise be difficult to remove.

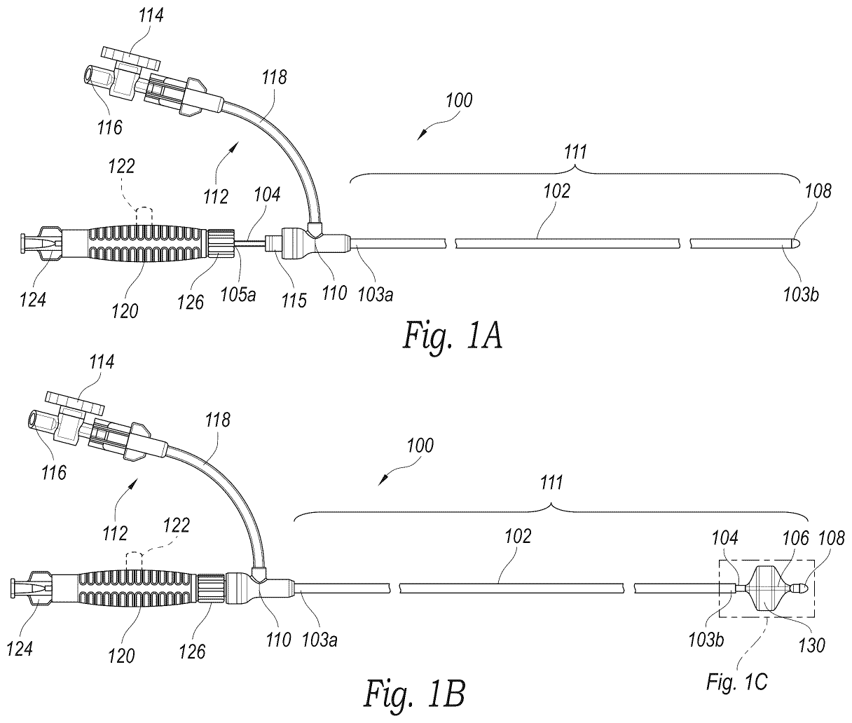

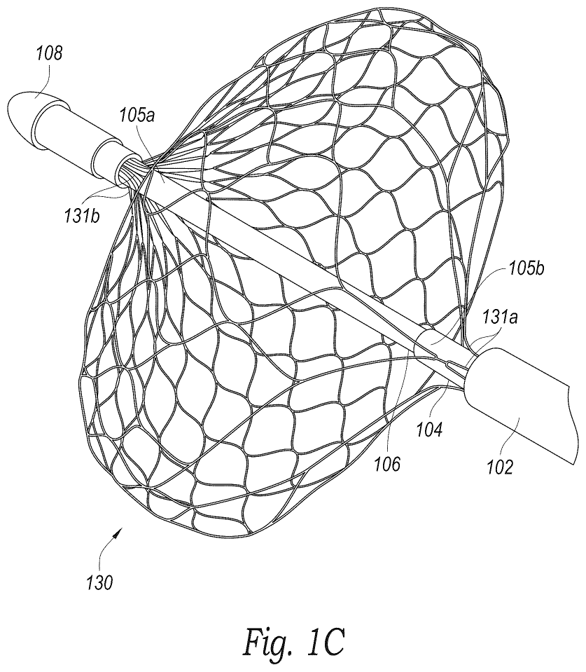

Although many of the embodiments are described below with respect to systems, devices, and methods for treating a pulmonary embolism, other applications and other embodiments in addition to those described herein are within the scope of the technology (e.g., intravascular procedures other than the treatment of emboli, intravascular procedures for treating cerebral embolism, intravascular procedures for treating deep vein thrombosis (DVT), etc.). Additionally, several other embodiments of the technology can have different configurations, states, components, or procedures than those described herein. Moreover, it will be appreciated that specific elements, substructures, advantages, uses, and/or other features of the embodiments described with reference to FIGS. 1-4F can be suitably interchanged, substituted or otherwise configured with one another in accordance with additional embodiments of the present technology. Furthermore, suitable elements of the embodiments described with reference to FIGS. 1-4F can be used as standalone and/or self-contained devices. A person of ordinary skill in the art, therefore, will accordingly understand that the technology can have other embodiments with additional elements, or the technology can have other embodiments without several of the features shown and described below with reference to FIGS. 1-4F.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” etc. are not meant to limit the referenced component to use in a specific orientation. It will be appreciated that such designations refer to the orientation of the referenced component as illustrated in the Figures; the systems and devices of the present technology can be used in any orientation suitable to the user.

FIGS. 1A and 1B are side views of a clot treatment or clot removal system 100 (“system 100”) configured in accordance with embodiments of the present technology. The system 100 is in a constrained/pre-deployment configuration in FIG. 1A, and the system 100 is in an expanded/deployed configuration in FIG. 1B. Referring to FIGS. 1A and 1B together, in the illustrated embodiment the system 100 includes a delivery catheter 102 (e.g., a tube, a shaft, etc.; which can also be referred to herein as an outer shaft) defining a lumen and having a proximal end portion 103a and a distal end portion 103b. The proximal end portion 103a of the delivery catheter 102 is coupled to a hub 110, such as a sealable hub, valve, etc. The lumen of the delivery catheter 102 can be fluidly coupled to a port assembly 112 via the hub 110.

In the illustrated embodiment, the port assembly 112 includes a fluid control device 114 fluidly coupled between (i) a port connector 116 (e.g., a Luer connector/fitting) and (ii) a tubing section 118 coupled to the hub 110 (e.g., to a branch or side port of the hub 110). The fluid control device 114 is actuatable to fluidly connect the lumen of the delivery catheter 102 to the port connector 116. In the illustrated embodiment, the fluid control device 114 is a stopcock while, in other embodiments, the fluid control device 114 can be a clamp, valve, and/or other suitable fluid control device. During a clot removal procedure using the system 100, various components (e.g., syringes, vacuum sources, etc.) can be coupled to the port connector 116 to remove fluid from and/or inject fluid into the lumen of the delivery catheter 102. For example, in some embodiments a syringe or other pressure source can be coupled to the port connector 116 and used to draw a vacuum while the fluid control device 114 is closed, and the fluid control device 114 can then be opened to instantaneously or nearly instantaneously apply the vacuum to the lumen of the delivery catheter 102 (e.g., to generate suction at the distal portion 103b for removing clot material). In other embodiments, a constant vacuum source (e.g., a pump) can be coupled to the port assembly 112 to provide constant aspiration of the lumen of the delivery catheter 102. In some embodiments, flushing fluid (e.g., saline) can be injected through the port assembly 112 to flush the lumen of the delivery catheter 102.

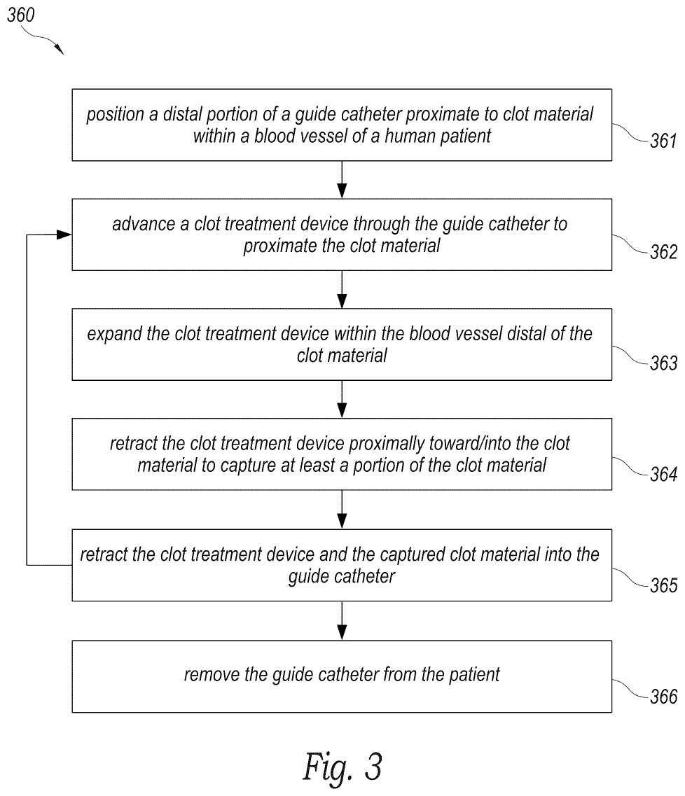

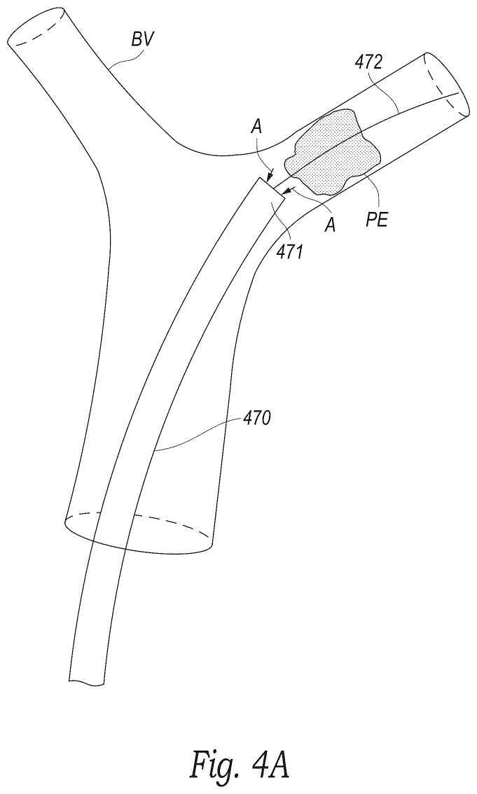

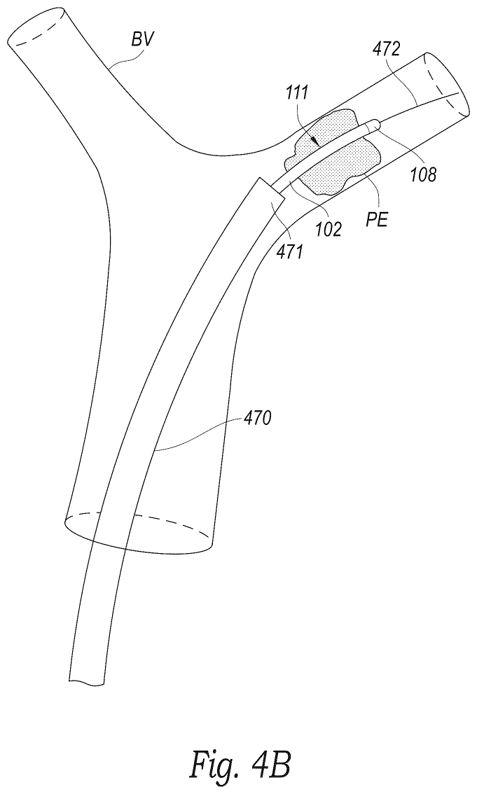

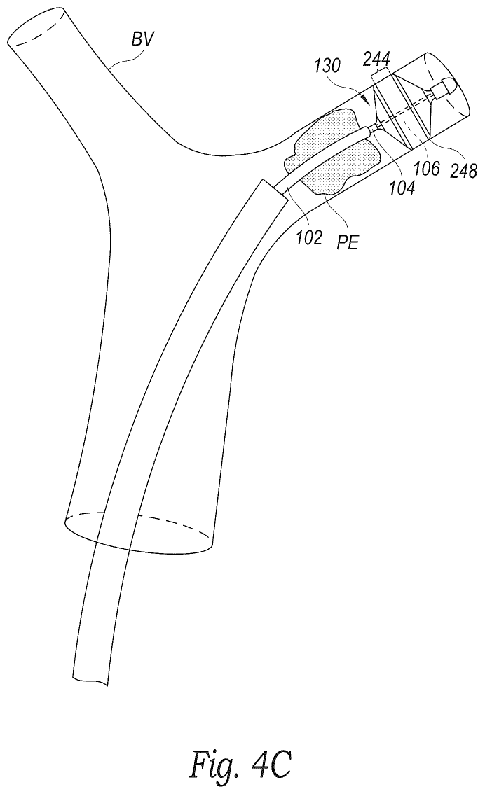

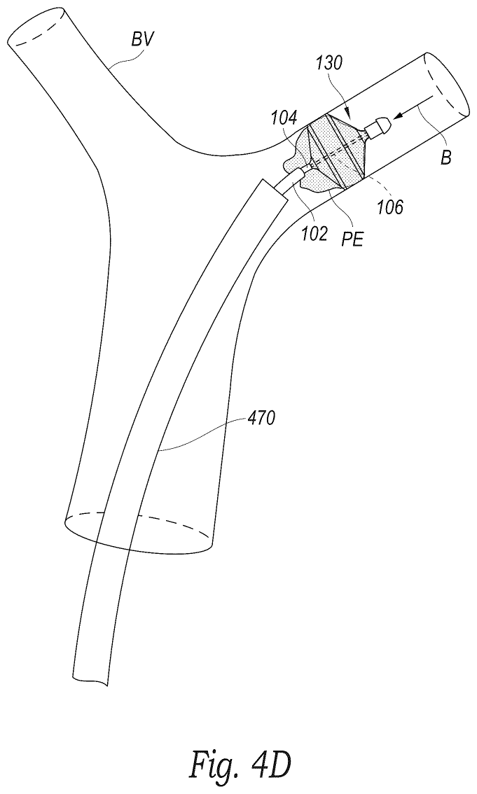

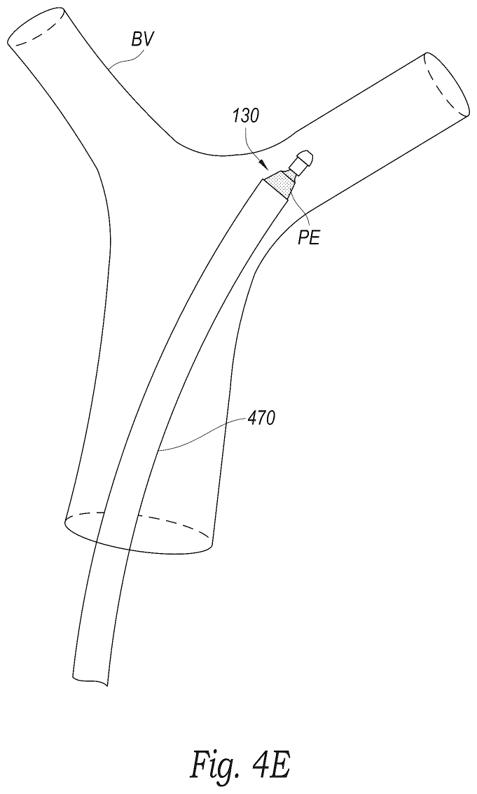

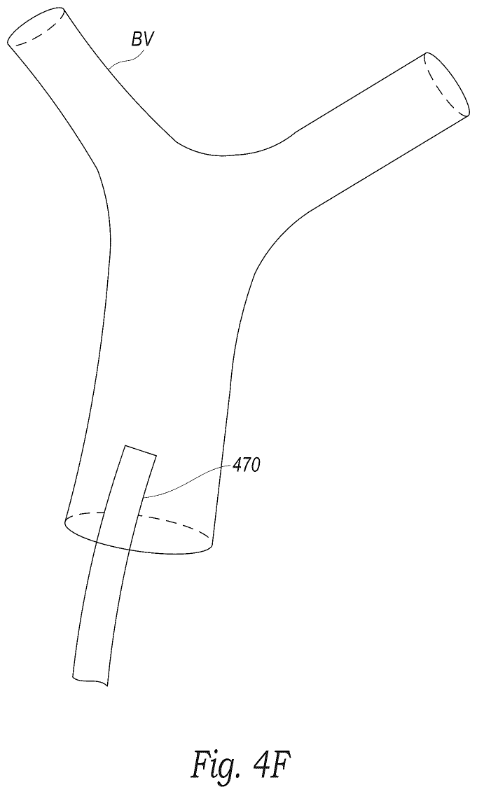

In the illustrated embodiment, the system 100 further includes an intermediate shaft 104 (e.g., a catheter, tube, etc.) extending at least partially through the lumen of the delivery catheter 102 and defining a lumen, and an inner shaft 106 (e.g., a catheter, tube, etc.) extending at least partially through the lumen of the intermediate shaft 104. Accordingly, in some embodiments the delivery catheter 102, the intermediate shaft 104, and the inner shaft 106 are coaxially aligned/arranged. The system 100 further includes a clot treatment device 130 coupled to the intermediate shaft 104 and the inner shaft 106. The delivery catheter 102, the intermediate shaft 104, the inner shaft 106, and the clot treatment device 130 can collectively be referred to as a treatment portion 111 (e.g., an insertion portion) of the system 100. As described in greater detail below with reference to FIGS. 3-4F, the treatment portion 111 is configured to be inserted through a guide catheter to position the clot treatment device 130 at a treatment site during a clot removal procedure.

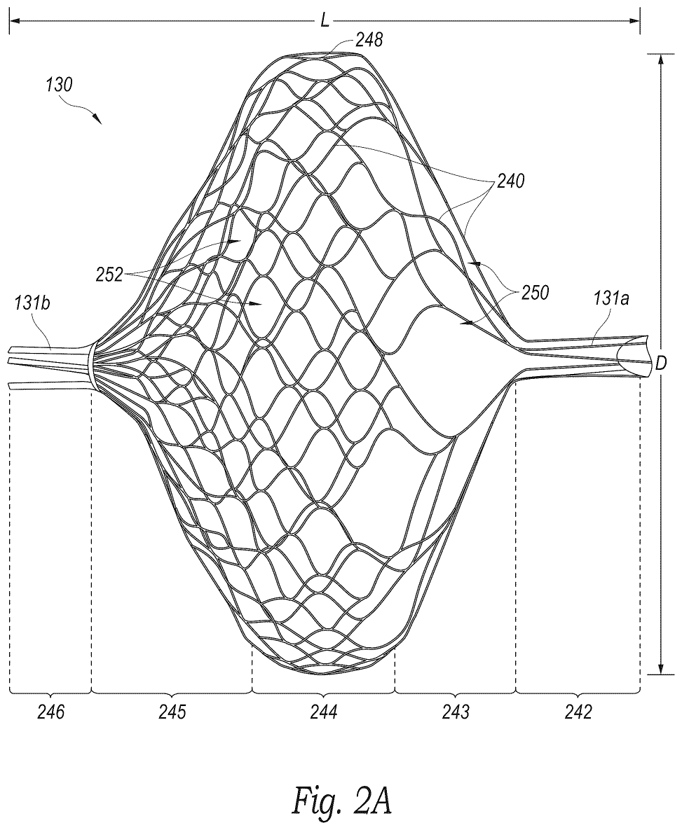

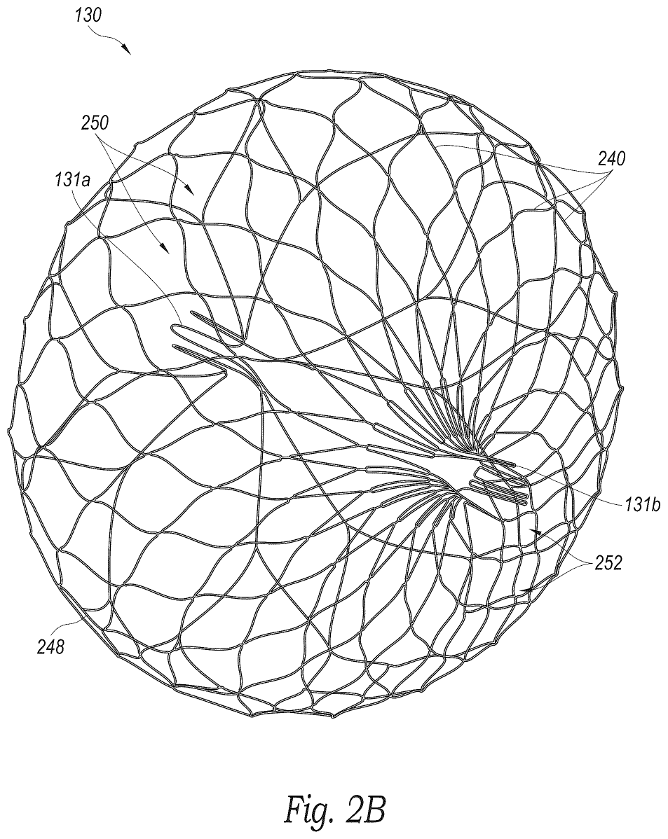

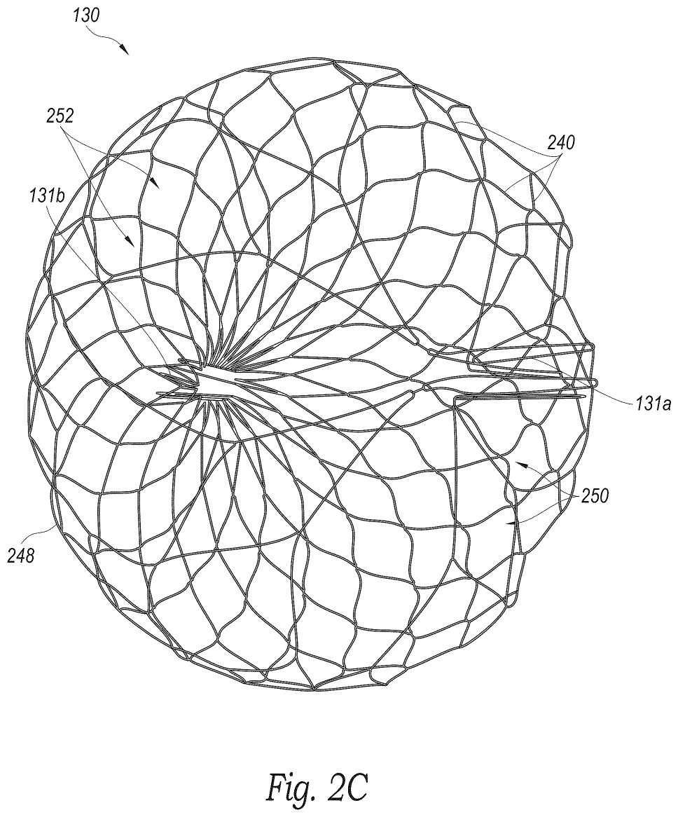

As described in greater detail below with reference to FIGS. 2A-2C, the clot treatment device 130 can be a self-expanding unitary structure comprising a plurality of interconnected struts. In the pre-deployment configuration shown in FIG. 1A, the clot treatment device 130 is constrained within the delivery catheter 102 and thus obscured. In the deployed configuration shown in FIG. 1B, the clot treatment device 130 extends past the distal end portion 103b of the delivery catheter 102 (e.g., a distal terminus of the delivery catheter 102) and is radially expanded.

FIG. 1C is an enlarged perspective view of a distal portion of the system 100 shown in FIG. 1B configured in accordance with an embodiment of the present technology. In the illustrated embodiment, the intermediate shaft 104 includes a distal end portion 105b coupled to a proximal portion 131a of the clot treatment device 130. In some embodiments, the proximal portion 131a of the clot treatment device 130 includes a plurality of struts that are gathered together and secured to the distal end portion 105b of the intermediate shaft 104. For example, the struts at the proximal portion 131a of the clot treatment device 130 can be secured to the outer surface of the intermediate shaft 104 via adhesives, fasteners, a hub or other device, etc. The inner shaft 106 includes a distal end portion 107 coupled to a distal portion 131b of the clot treatment device 130. In some embodiments, the distal portion 131b of the clot treatment device 130 includes a plurality of struts that are gathered together and secured to the distal end portion 107 of the inner shaft 106 via a friction fit, pressure fit, etc., between the inner shaft 106 and a distal tip 108 (e.g., an atraumatic tip). In other embodiments, the struts at the distal portion 131b of the clot treatment device 130 can be secured to the outer surface of the inner shaft 106 via adhesives, fasteners, a hub or other device, etc.

Referring again to FIGS. 1A and 1B together, the intermediate shaft 104 includes a proximal end portion 105a coupled to the handle 120 (e.g., to a distal portion of the handle 120) to operably couple the handle 120 to the clot treatment device 130. Accordingly, the intermediate shaft 104 extends between and operably couples the handle 120 and the clot treatment device 130. In some embodiments, a proximal end portion of the inner shaft 106 (obscured in FIGS. 1A and 1B) is not coupled to any portion of the system 100 and floats within the lumen of the intermediate shaft 104. In one aspect of the present technology, this arrangement allows the inner shaft 106 to move relative to the intermediate shaft 104 in response to external forces on the clot treatment device 130, thereby allowing the clot treatment device 130 to elongate/shorten longitudinally and to correspondingly radially compress/expand. In other embodiments, the proximal end portion of the inner shaft 106 can be coupled to an actuation mechanism 122 (shown in dashed lines in FIGS. 1A and 1B) of the handle 120. The actuation mechanism 122 can be configured to drive the inner shaft 106 proximally and/or distally to shorten and/or elongate, respectively, the clot treatment device 130. More specifically, in some embodiments distal movement of the actuation mechanism 122 relative to the handle 120 can move the inner shaft 106 distally relative to the intermediate shaft 104 to lengthen and radially compress the clot treatment device 130, while proximal movement of the actuation mechanism 122 relative to the handle 120 can move the inner shaft 106 proximally relative to the intermediate shaft 104 to shorten and radially expand the clot treatment device 130.

In the illustrated embodiment, the handle 120 further includes a proximal hub 124, such as a Luer hub, configured to receive a guidewire (not shown) therethrough. The handle 120, the inner shaft 106, and the tip 108 can together define a lumen for receiving the guidewire therethrough. In some embodiments, the guidewire can have a diameter of about 0.035 inch, about 0.018 inch, less than about 0.1 inch, less than about 0.05 inch, etc. In some embodiments, the handle 120 further includes a lock feature 126 such as, for example, a spinlock or a push-in-and-turn lock. The lock feature 126 is configured to selectively engage (e.g., lockingly engage) with a mating feature 115 of the hub 110. Locking the handle 120 to the hub 110 via the lock feature 126 and the mating feature 115 secures the position of the intermediate shaft 104 relative to the delivery catheter 102. In the illustrated embodiment, the intermediate shaft 104 is longer than the delivery catheter 102 such that a portion of the intermediate shaft 104 and the clot treatment device 130 extend distally from the distal end portion 103b of the delivery catheter 102 when the handle 120 is lockingly engaged with the hub 110.

To deploy the clot treatment device 130 from the pre-deployment configuration (FIG. 1A) to the deployed configuration (FIG. 1B), an operator can move the handle 120 distally toward the hub 110 and/or can move the hub 110 toward the handle 120. This movement advances the intermediate shaft 104 distally through the delivery catheter 102 and pushes the clot treatment device 130 distally out of the delivery catheter 102. The clot treatment device 130 can self-expand as it is released from the lumen of the delivery catheter 102. When the handle 120 abuts the hub 110, the operator can actuate the lock feature 126 to secure the position of the intermediate shaft 104 relative to the delivery catheter 102 to, for example, maintain the clot treatment device 130 in the deployed configuration.

In some embodiments, proximal movement of the handle 120 and/or distal movement of the hub 110 (e.g., from the position shown in FIG. 1B to the position shown in FIG. 1A) can retract the clot treatment device 130 back into the delivery catheter 102. That is, in some embodiments the clot treatment device 130 can be resheathed within the delivery catheter 102. In such embodiments, the clot treatment device 130 can be repeatedly expanded and then retracted and compressed into the delivery catheter 102. In some embodiments, the tip 108 is configured (e.g., sized and shaped) to abut the distal end portion 103b of the delivery catheter 102 in the pre-deployment configuration (FIG. 1A). This can inhibit or even prevent the clot treatment device 130 from being pulled fully through the delivery catheter 102 and, in some embodiments, can substantially seal the lumen of the delivery catheter 102. In other embodiments, the tip 108 is sized and shaped to allow the tip 108—and thus the entire clot treatment device 130—to be retracted through the delivery catheter 102.

FIGS. 2A-2C are a side view, a proximally-facing perspective view, and a distally-facing perspective view, respectively, of the clot treatment device 130 in the expanded configuration in accordance with embodiments of the present technology. Referring to FIGS. 2A-2C together, the clot treatment device 130 comprises a plurality of struts 240 that together define a plurality of first cells 250 (e.g., interstices, pores, openings, etc.) and a plurality of second cells 252. The struts 240 can have a variety of shapes and sizes and, in some embodiments, the struts 240 can have a thickness and/or diameter between about 0.0125-0.150 inch, between about 0.075-0.125 inch, between about 0.090-0.150 inch, and/or other dimensions. In general, the struts 240 together form a unitary structure that is configured to engage, capture, disrupt, and/or separate a portion of a thrombus (e.g., a vascular thrombus) from a blood vessel containing the thrombus.

In the illustrated embodiment, (i) the first cells 250 generally face proximally while the second cells 252 generally face distally, and (ii) the first cells 250 are larger than the second cells 252. As best seen in FIG. 2A, the clot treatment device 130 includes (i) a first region 242 including the proximal portion 131a, (ii) a second region 243 distal of the first region 242, (iii) a third (e.g., central) region 244 distal of the second region 243, (iv) a fourth region 245 distal of the third region 244, and (v) a fifth region 246 distal of the fourth region 245 and including the distal portion 131b. In the illustrated embodiment, the struts 240 are gathered together (e.g. positioned proximate one another) at the first and fifth regions 242, 246 to facilitate their connection to the intermediate and inner shafts 104, 106, respectively, as shown in FIG. 1C. The second region 243 can have a generally conical shape that tapers (e.g., radially narrows) in the proximal direction. Similarly, the fourth region 245 can have a generally conical shape that tapers in the distal direction. The third region 244 can have a generally tubular/cylindrical shape including, for example a generally flat outer strut surface/boundary 248. Moreover, in the illustrated embodiment the first and second regions 242, 243 have fewer of the struts 240 than the fourth and fifth regions 245, 246 to thereby define the larger first cells 250. Conversely, the fourth and fifth regions 245, 246 have more of the struts 240 than the first and second regions 242, 243 to thereby define the smaller second cells 252. The third region 244 can be a transition region in which the number of the struts 240 increases in the proximal direction (e.g., toward the fourth region 245) such that some of the first cells 250 abut some of the second cells 252 in the third region 244. In other embodiments, the first cells 250 can be formed only in the second region 243, can occupy the entire third region 244, can extend into the fourth region 245, etc.

In some embodiments, the clot treatment device 130 is made from a shape memory material such as a shape memory alloy and/or a shape memory polymer. For example, the clot treatment device 130 can comprise nitinol and/or a nitinol alloy. Similarly, the clot treatment device 130 can be made using a variety of techniques including welding, laser welding, cutting, laser cutting, expanding, etc. For example, in some embodiments the clot treatment device 130 can first be laser cut from a piece of nitinol (e.g., a nitinol tube), and then further shaped using a heat setting process such that the clot treatment device 130 has the illustrated shape in the expanded configuration. For example, as is known in the art of heat setting nitinol structures, a fixture, mandrel, or mold may be used to hold the clot treatment device 130 in its desired configuration, and then the clot treatment device 130 can be subjected to an appropriate heat treatment such that the struts 240 of the clot treatment device 130 assume or are otherwise shape-set to the outer contour of the mandrel or mold. The heat setting process may be performed in an oven or fluidized bed, as is well-known. Therefore, the heat setting process can impart a desired shape, geometry, bend, curve, serration, scallop, void, hole, etc., in the super-elastic and/or shape memory material or materials used to form the clot treatment device 130. Accordingly, the clot treatment device 130 may be radially constrained without plastic deformation and will self-expand on release of the radial constraint.

In general, the size of the clot treatment device 130 can be selected based on the size (e.g., diameter) of the blood vessel from which thrombus is to be extracted. In some embodiments, in a fully-expanded configuration unconstrained within a vessel, the clot treatment device 130 can have a length L (FIG. 2A) of between about 0.025-1.50 inches, between about 0.70-1.15 inches, etc. In some embodiments, in the fully-expanded position unconstrained within a vessel, the clot treatment device 130 can have a maximum diameter D (FIG. 2A; e.g., at the third region 244) of between about 0.025-1.5 inches, between about 0.71-1.34 inches, etc.

The clot treatment device 130 is configured (e.g., shaped, sized, angled, formed, etc.) to engage, disrupt, and/or capture clot material from within a blood vessel when the clot treatment device 130 is retracted through/against the clot material in the expanded configuration. For example, as described in greater detail below with reference to FIGS. 3-4F, the clot treatment device 130 can be withdrawn proximally through/against the clot material. In one aspect of the present technology, the larger first cells 250 are configured to receive the clot material therethrough as the clot treatment device 130 is pulled against the clot material, and the smaller second cells 252 (and associated struts 240) are configured to retain the clot material within the clot treatment device 130. In another aspect of the present technology, the clot treatment device 130 has sufficient radial stiffness (e.g., at the third region 244) to inhibit the clot treatment device 130 from slipping (e.g., not engaging) the clot material when the clot treatment device 130 is pulled against the clot material. Accordingly, the clot treatment device 130 can be used to capture/disrupt adhered, organized, and/or chronic clots. In some embodiments, portions of the struts 240 (e.g., at the second region 243) can be sharpened and/or can include a cutting element (e.g., a knife or knife edge) attached thereto or otherwise integrated with to further facilitate disruption/cutting of the clot material.

FIG. 3 is a flow diagram of a process or method 360 for operating the system 100 to remove clot material from within a blood vessel (e.g., a pulmonary blood vessel) of a patient (e.g., a human patient) in accordance with an embodiment the present technology. FIGS. 4A-4F are schematic illustrations of a distal portion of the system 100 inserted through a guide catheter 470 during a procedure to remove clot material PE from a blood vessel BV of a patient in accordance with embodiments of the present technology. Although some features of the method 360 are described in the context of the embodiments shown in FIGS. 4A-4F for the sake of illustration, one skilled in the art will readily understand that the method 360 can be carried out using other suitable systems and/or devices described herein.

With reference to FIGS. 3 and 4A, at block 361, the method 360 can include positioning a distal portion 471 of the guide catheter 470 proximate to the clot material PE within the blood vessel BV (e.g., at a treatment site). In the illustrated embodiment, a distal terminus of the guide catheter 470 is positioned proximate to a proximal portion of the clot material PE. However, in other embodiments the distal terminus of the guide catheter 470 can be positioned at least partially within the clot material PE, or the distal terminus of the guide catheter 470 can be positioned distal of the clot material PE. Access to the blood vessel BV can be achieved through the patient's vasculature, for example, via the femoral vein. In some embodiments, such as when the blood vessel BV is a pulmonary blood vessel, an introducer (e.g., a Y-connector with a hemostasis valve; not shown) is connected to the guide catheter 470 and can be partially inserted into the femoral vein. A guidewire 472 can be guided into the femoral vein through the introducer and navigated through the right atrium, the tricuspid valve, the right ventricle, the pulmonary valve, and into the main pulmonary artery. Depending on the location of the clot material PE, the guidewire 472 can be guided to one or more of the branches of the right pulmonary artery and/or the left pulmonary artery. In some embodiments, the guidewire 472 can be extended entirely or partially through the clot material PE. In other embodiments, the guidewire 472 can be extended to a location just proximal of the clot material PE. After positioning the guidewire 472, the guide catheter 470 can be placed over the guidewire 472 and advanced to the position proximate to the clot material PE as illustrated in FIG. 4A.

In some embodiments, a pressure source can be coupled to the guide catheter 470 and used to aspirate the lumen of the guide catheter 470 to, for example, generate suction (e.g., as indicated by arrows A) to suck/draw all or a portion of the clot material PE into the guide catheter 470. For example, in some embodiments a vacuum can be pre-charged (e.g., in a syringe fluidly coupled to the lumen of the guide catheter 470) and the vacuum can be applied to the lumen of the guide catheter 470 to instantaneously or nearly instantaneously generate suction at the distal portion 471 of the guide catheter 470 (e.g., to generate a suction pulse at the distal portion 471 of the guide catheter 470). Specific details of such methods and associated devices are disclosed in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety.

However, even where suction is applied to remove/dislodge the clot material PE from the blood vessel BV, the suction may not be enough to dislodge/disrupt all the clot material PE. For example, many chronic (e.g., organized) clots can strongly adhere to the walls of the blood vessel BV—making it difficult to remove them. In one aspect of the present technology, the system 100 can be inserted through the guide catheter 470 before, during, and/or after suction is applied via the guide catheter 470 to engage, disrupt, and/or capture the clot material PE—even where the clot material PE is strongly adhered within the blood vessel BV.

For example, with reference to FIGS. 3 and 4B, at block 362, the method 360 can include advancing the clot treatment device 130 (compressed within the delivery catheter 102 and thus obscured in FIG. 4B) through the guide catheter 470 to proximate the clot material PE. More specifically, the treatment portion 111 of the system 100 can be advanced through the guide catheter 470 in the compressed pre-deployment configuration until the tip 108 is positioned (i) distal of the distal portion 471 of the guide catheter 470 and (ii) distal of the clot material PE within the blood vessel BV. In other embodiments, the tip 108 can be positioned within the clot material PE. In some embodiments, the treatment portion 111 can be advanced over the guidewire 472 while, in other embodiments, the guidewire 472 can be omitted.

With reference to FIGS. 3 and 4C, at block 363, the method 360 can include moving the clot treatment device 130 from the compressed pre-deployment configuration to the expanded deployed configuration such that the clot treatment device 130 is expanded distal and/or partially within the clot material PE. For example, as described in detail above with reference to FIGS. 1A and 1B, an operator of the system 100 can advance the handle 120 distally toward the hub 110 and/or retract the hub 110 toward the handle 120 to move the intermediate shaft 104 relative to the delivery catheter 102 to advance the clot treatment device 130 out of the delivery catheter 102, thereby allowing the clot treatment device 130 to expand (e.g., self-expand) within the blood vessel BV. In the illustrated embodiment, the clot treatment device 130 (e.g., the outer strut surface 248 of the third region 244) contacts (e.g., engages, apposes, etc.) the wall of the blood vessel BV. In some embodiments, the clot treatment device 130 is oversized relative to the blood vessel BV such that the clot treatment device 130 exerts a radially outward force on the wall of the blood vessel BV. In other embodiments, the clot treatment device 130 can be sized such that it does not contact the walls of the blood vessel BV.

With reference to FIGS. 3 and 4D, at block 364, the method 360 can include retracting the clot treatment device 130 proximally (e.g., in the direction of arrow B) into/toward the clot material PE. More specifically, with reference to FIGS. 1A and 1B, the operator can pull the entire system 100 proximally (e.g., by gripping the hub 110) to retract the treatment portion 111 through the lumen of the guide catheter 470. As the clot treatment device 130 is retracted, the clot treatment device 130 engages the clot material PE to capture/disrupt the clot material PE. For example, the clot material PE can enter through the first cells 250 (FIGS. 2A-2C) and be retained within the clot treatment device 130 by the smaller second cells 252 (FIGS. 2A-2C). In one aspect of the present technology, the clot treatment device 130 can shear the clot material PE from the wall of the blood vessel BV even where the clot material PE is strongly adhered to the wall of the blood vessel BV.

In some embodiments, where the inner shaft 106 floats within the lumen of the intermediate shaft 104, the length L (FIG. 2A) of the clot treatment device 130 can increase as the clot treatment device 130 is pulled into/against the clot material PE and the intermediate shaft 104 moves proximally relative to the inner shaft 106. In other embodiments, where the system 100 includes the actuation mechanism 122, the operator can actuate the actuation mechanism 122 to increase the longitudinal and/or radial stiffness of the clot treatment device 130 by locking or substantially locking the relative position of the intermediate and inner shafts 104, 106.

With reference to FIGS. 3 and 4E, at block 365, the method 360 can include retracting the clot treatment device 130 and the captured clot material PE into the lumen of the guide catheter 470. In some embodiments, the clot treatment device 130 can be fully removed from the guide catheter 470. In some embodiments, if any of the clot material PE remains in the blood vessel BV, the clot treatment device 130 can be cleaned and blocks 362-365 can be repeated to capture the remaining clot material PE. Alternatively, a new clot treatment device 130 can be reinserted through the guide catheter 470 to capture the remaining clot material PE. In some embodiments, the clot treatment device 130 can break apart the clot material PE without necessarily capturing the clot material PE and, after or during retraction of the clot treatment device 130, aspiration can be applied to the guide catheter 470 to suck the remaining clot material PE into the guide catheter 470. Finally, with reference to FIGS. 3 and 4F, at block 366, the method 360 can include removing the guide catheter 470 from the blood vessel BV and from the patient after a sufficient portion of the clot material is removed from the patient.

Several aspects of the present technology are set forth in the following additional examples:

1. A clot treatment system, comprising:
an outer catheter defining a lumen;
an inner catheter positioned at least partially in the lumen of the outer catheter; and
a clot treatment device including a plurality of interconnected struts forming a unitary structure movable between a compressed configuration and an expanded configuration, wherein in the expanded configuration the unitary structure includes—a proximal connection region coupled to the outer catheter;
   a proximal conical region extending from the proximal connection region, wherein a first portion of the struts form first cells in the proximal conical region;
   a cylindrical region extending from the proximal conical region;
   a distal conical region extending from the cylindrical region, wherein a second portion of the struts from second cells in the distal conical region, and wherein the second cells are smaller than the first cells; and
   a distal connection region extending from the distal conical region and coupled to the inner catheter.

2. The clot treatment system of example 1 wherein the inner catheter has (a) a distal end portion coupled to the distal connection region of the clot treatment device and (b) a proximal end portion configured to float within the lumen of the outer catheter.

3. The clot treatment system of example 1 or example 2 wherein the inner and outer catheters are configured to receive a guidewire therethrough.

4. The clot treatment system of any one of examples 1-3, further comprising a handle coupled to a proximal end portion of the outer catheter, wherein the handle includes an actuation mechanism coupled to a proximal end portion of the inner catheter, and wherein actuation of the actuation mechanism is configured to translate the inner catheter relative to the outer catheter to longitudinally compress or longitudinally elongate the clot treatment device.

5. The clot treatment system of any one of examples 1-4, further comprising:
a delivery catheter defining a lumen; and
a handle coupled to a proximal end portion of the outer catheter and movable between a first position and a second position relative to the delivery catheter, wherein—
   in the first position, the clot treatment device is constrained within the lumen of the delivery catheter in the compressed configuration, and
   in the second position, the clot treatment device is positioned distal of the lumen in the expanded configuration.

6. The clot treatment system of example 5, further comprising a hub coupled to a proximal end portion of the delivery catheter, wherein the handle includes a lock feature configured to secure the handle to the hub in the second position.

7. The clot treatment system of example 5 or example 6 wherein the handle, the delivery catheter, the outer catheter, and the inner catheter are configured to receive a guidewire therethrough.

8. The clot treatment system of any one of examples 1-7 wherein, in the expanded configuration, the cylindrical region has a diameter of between about 0.71 inch to about 1.34 inches.

9. The clot treatment system of any one of examples 1-8 wherein the struts of the clot treatment device are configured to self-expand from the compressed configuration to the expanded configuration when unconstrained.

10. The clot treatment system of any one of examples 1-9 wherein the struts of the clot treatment device include a shape memory material.

11. The clot treatment system of any one of examples 1-10 wherein the unitary structure includes (a) a first number of the struts in the proximal conical region and (b) a second number of the struts in the distal conical region that is greater than the first number of struts.

12. A method of clot removal, the method comprising:
positioning a distal portion of a guide catheter proximate to clot material within a blood vessel of a human patient;
advancing a clot treatment device through the guide catheter to proximate the clot material;
expanding the clot treatment within the blood vessel distal of the clot material, wherein the clot treatment device includes a plurality of interconnected struts forming a unitary structure having a proximal portion and a distal portion, wherein the struts form a plurality of first cells in the proximal portion and a plurality of second cells in the distal portion, and wherein the first cells are larger than the second cells;
generating suction at the distal portion of the guide catheter; and
proximally retracting the clot treatment device through the clot material.

13. The method of example 12 wherein advancing the clot treatment device through the guide catheter includes advancing the clot treatment device over a guidewire.

14. The method of example 12 or example 13 wherein the proximal portion of the unitary structure is coupled to an outer catheter extending at least partially through the guide catheter, and wherein the distal portion of the unitary structure is coupled to an inner catheter extending at least partially through the outer catheter.

15. The method of example 14 wherein advancing the clot treatment device through the guide catheter includes advancing the clot treatment device over a guidewire extending through the guide, outer, and inner catheters.

16. The method of any one of examples 12-15 wherein generating suction at the distal portion of the guide catheter includes generating suction, before proximally retracting the clot treatment device, to aspirate a first portion of the clot material into the guide catheter.

17. The method of example 16 wherein proximally retracting the clot treatment device includes proximally retracting the clot treatment device through a second portion of the clot material remaining in the blood vessel to capture the second portion of the clot material.

18. The method of any one of examples 12-17 wherein proximally retracting the clot treatment device through the clot material includes capturing at least a portion of the clot material, and wherein the method further comprises retracting the clot treatment device and the captured clot material into the guide catheter.

19. A clot treatment system, comprising:
an outer shaft defining a lumen;
an inner shaft positioned at least partially in the lumen of the outer shaft; and
a plurality of interconnected struts forming a unitary structure having a proximal portion and a distal portion, wherein the proximal portion is coupled to the outer shaft, wherein the distal portion is coupled to the inner shaft, and wherein the struts form a plurality of first cells in the proximal portion and a plurality of second cells in the distal portion, and wherein the first cells are larger than the second cells.

20. The clot treatment system of example 12 wherein the outer shaft and the inner shaft are configured to receive a guidewire therethrough.

21. A clot treatment device, comprising:
a plurality of interconnected struts forming a unitary structure movable between a compressed configuration and an expanded configuration, wherein in the expanded configuration the unitary structure includes—
   a proximal connection region;
   a proximal conical region extending from the proximal connection region, wherein a first portion of the struts form first cells in the proximal conical region;
   a cylindrical region extending from the proximal conical region;
   a distal conical region extending from the cylindrical region, wherein a second portion of the struts from second cells in the distal conical region, and wherein the second cells are smaller than the first cells; and
   a distal connection region extending from the distal conical region.

22. The clot treatment device of example 21, further comprising:
a first shaft coupled to the proximal connection region and defining a lumen; and
a second shaft coupled to the distal connection region and extending at least partially through the lumen of the first shaft.

23. The clot treatment device of example 21 or example 22 wherein the second shaft has (a) a distal end portion coupled to the distal connection region and (b) a proximal end portion configured to float within the lumen of the first shaft.

24. The clot treatment device of any one of examples 21-23 wherein the struts are configured to self-expand from the compressed configuration to the expanded configuration when unconstrained.

25. The clot treatment device of any one of examples 21-24 wherein the struts are made from a shape memory material.

26. A clot treatment device, comprising:
a plurality of interconnected struts forming a unitary structure having a proximal portion and a distal portion, wherein the struts form a plurality of first cells in the proximal portion and a plurality of second cells in the distal portion, and wherein the first cells are larger than the second cells.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-20. (canceled)

21. A system for treating clot material in the vasculature of a patient, the system comprising:
a guide catheter defining a lumen and having a distal end portion, wherein the catheter is configured to be intravascularly advanced through the vasculature of the patient such that the distal end portion of the guide catheter is positioned proximate to the clot material;
a syringe having a barrel, wherein the syringe is actuatable to generate vacuum pressure in the barrel, wherein the barrel is configured to store the vacuum pressure, and wherein the syringe is configured to be fluidly coupled to the lumen of the guide catheter to apply the vacuum pressure stored in the barrel to the lumen to aspirate at least a first portion of the clot material into the lumen; and
a clot treatment comprising a delivery catheter, a shaft, and an expandable member having a proximal end portion secured to the shaft, wherein—
the expandable member includes a plurality of interconnected struts forming a unitary structure movable between a compressed configuration within the delivery catheter and an expanded configuration outside the delivery catheter,
in the expanded configuration, the unitary structure comprises (a) a proximal conical region extending from the proximal end portion, (b) a cylindrical region extending from the proximal conical region, and (c) a distal conical region extending from the cylindrical region to a distal end portion of the expandable member,
the clot treatment device is configured to be inserted through the guide catheter with the expandable member in the compressed configuration within the delivery catheter,
the clot treatment device is configured to be moved to the expanded configuration outside the delivery catheter distal to the guide catheter, and
the expandable member is configured to be retracted proximally in the expanded configuration to capture at least a second portion of the clot material that remains in the blood vessel after aspiration of the first portion of the clot material.

22. The system of claim 21 wherein the clot treatment device further comprises a distal tip coupled to the distal end portion of the expandable member.

23. The system of claim 22 wherein the distal tip is configured to abut a distal end of the delivery catheter when the expandable member is in the compressed configuration within the delivery catheter.

24. The system of claim 21 wherein the struts of the expandable member form first cells in the proximal conical region and second cells in the distal conical region, and wherein the first cells are larger than the second cells.

25. The system of claim 21 wherein the shaft is a first shaft, and wherein the clot treatment device further comprises a second shaft extending through the first shaft and coupled to the distal end portion of the expandable member.

26. The system of claim 21 wherein the syringe is fluidly coupled to the lumen of the guide catheter via a valve, wherein the valve is movable between (a) a first position that inhibits fluid flow from the lumen of the guide catheter to the barrel and (b) a second position that permits fluid flow from the lumen of the guide catheter to the barrel.

27. The system of claim 26 wherein the syringe is configured to be actuated to generate the vacuum pressure in the barrel with the valve in the first position, and wherein the valve is movable from the first position to the second position while the vacuum pressure is stored in the barrel, thereby applying the vacuum pressure to the lumen of the guide catheter to aspirate the first portion of the clot material into the lumen.

28. The system of claim 21 wherein the expandable member is further configured to be retracted in the expanded configuration into the lumen of the guide catheter with the captured second portion of the clot material.

29. The system of claim 21 wherein the struts of the expandable member are configured to self-expand from the compressed configuration to the expanded configuration when the expandable member is outside the delivery catheter.

30. The system of claim 21 wherein the struts of the clot treatment device include a shape memory material.

31. The system of claim 21 wherein the syringe is a vacuum-pressure locking syringe.

32. The system of claim 21 wherein the clot material comprises a pulmonary embolism.

33. The clot treatment system of claim 21 wherein the clot material comprises a deep vein thrombosis.

34. A system for treating clot material in the vasculature of a patient, the system comprising:
a guide catheter defining a lumen and having a distal end portion, wherein the catheter is configured to be intravascularly advanced through the vasculature of the patient such that the distal end portion of the guide catheter is positioned proximate to the clot material;
a pressure source configured to generate vacuum and to store the vacuum pressure, wherein the pressure source is configured to be fluidly coupled to the lumen of the guide catheter to apply the vacuum pressure stored therein to the lumen to aspirate at least a first portion of the clot material into the lumen; and
a clot treatment comprising a delivery catheter, a shaft, and an expandable member having a proximal end portion secured to the shaft, wherein—
catheter, a shaft, and an expandable member having a proximal end portion secured to the shaft, and wherein—
the expandable member includes a plurality of interconnected struts forming a unitary structure movable between a compressed configuration within the delivery catheter and an expanded configuration outside the delivery catheter,
in the expanded configuration, the unitary structure comprises (a) a proximal conical region extending from the proximal end portion, (b) a cylindrical region extending from the proximal conical region, and (c) a distal conical region extending from the cylindrical region to a distal end portion of the expandable member,
the clot treatment device is configured to be inserted through the guide catheter with the expandable member in the compressed configuration within the delivery catheter,
the clot treatment device is configured to be moved to the expanded configuration outside the delivery catheter distal to the guide catheter, and
the expandable member is configured to be retracted proximally in the expanded configuration to capture at least a second portion of the clot material that remains in the blood vessel after aspiration of the first portion of the clot material.

35. The system of claim 34 wherein the clot treatment device further comprises a distal tip coupled to the distal end portion of the expandable member, and wherein the distal tip is configured to abut a distal end of the delivery catheter when the expandable member is in the compressed configuration within the delivery catheter.

36. The system of claim 34 wherein the syringe is fluidly coupled to the lumen of the guide catheter via a valve, wherein the valve is movable between (a) a first position that inhibits fluid flow from the lumen of the guide catheter to the barrel and (b) a second position that permits fluid flow from the lumen of the guide catheter to the barrel, wherein the syringe is configured to be actuated to generate the vacuum pressure in the barrel with the valve in the first position, and wherein the valve is movable from the first position to the second position while the vacuum pressure is stored in the barrel, thereby applying the vacuum pressure to the lumen of the guide catheter to aspirate the first portion of the clot material into the lumen.

37. The system of claim 34 wherein the clot material comprises a pulmonary embolism or deep vein thrombosis.

38. The system of claim 34 wherein the expandable member is further configured to be retracted in the expanded configuration into the lumen of the guide catheter with the captured second portion of the clot material.

39. The system of claim 34 wherein the struts of the expandable member are configured to self-expand from the compressed configuration to the expanded configuration when the expandable member is outside the delivery catheter.
